(19) United States
(12) Patent Application Publication
(10) Pub. No.: US 2014/0329892 A1
(43) Pub. Date: Nov. 6, 2014

(54) ADMINISTRATION OF A BIS(THIOHYDRAZIDE AMIDE) COMPOUND FOR TREATING CANCERS

(71) Applicant: Synta Pharmaceuticals Corp., Lexington, MA (US)

(72) Inventors: Jim Sang, Wakefield, MA (US); Yumiko Wada, Tokyo (JP)

(73) Assignee: SYNTA PHARMACEUTICALS CORP., Lexington, MA (US)

(21) Appl. No.: 14/357,022

(22) PCT Filed: Nov. 9, 2012
(86) PCT No.: PCT/US2012/064449

§ 371 Date: May 8, 2014

Related U.S. Application Data
(60) Provisional application No. 61/558,411, filed on Nov. 10, 2011.
(60) Provisional application No. 61/558,412, filed on Nov. 10, 2011.

(51) Int. Cl. A61K 31/30; C07C 327/56; A61K 31/337

(52) U.S. Cl. CPC A61K 31/30; A61K 31/337; C07C 327/56
USPC 514/449; 514/499

(57) ABSTRACT

Administration of a bis(thiohydrazide amide) compound is found to be surprisingly effective at treating subjects with cancer. Methods of treating a subject with cancer comprising continuously administering a bis(thiohydrazide amide) compound, or administering a bis(thiohydrazide amide) compound such that a constant concentration of the compound is achieved in the subject, are disclosed.

### RELATED APPLICATIONS

This application claims priority to U.S. Provisional Application No. 61/558,411, filed on Nov. 10, 2011, and to U.S. Provisional Application No. 61/558,412, filed on Nov. 10, 2011, the entire contents of each of which are hereby incorporated herein by reference.

### BACKGROUND OF THE INVENTION

Elesclomol is a bis(thiohydrazide amide) compound of the following structural formula:

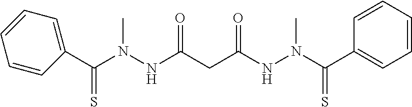 [SMILES: CN(NC(=O)CC(=O)NN(C)C(=S)c1ccccc1)C(=S)c1ccccc1]

Elesclomol is used for treating cancer and is known for its ability to cause oxidative stress in cells by elevating the levels of reactive oxygen species (ROS) beyond the threshold compatible with cell survival. Elesclomol readily forms a copper (Cu) chelate at a 1:1 molar ratio, and formation of this bioactive elesclomol-Cu complex is a rate-limiting step for its activity. Methods for improving efficacy of elesclomol would be highly desirable.

### SUMMARY OF THE INVENTION

It has been found that elesclomol, when administered continuously as a single agent, or when administered as a single agent in such a way as to maintain a constant level of elesclomol in the subject's body, is surprisingly effective at treating cancer. Based on the above discoveries, methods of treating a subject with cancer by continuously administering elesclomol to the subject are described herein.

In some embodiments, the invention provides a method of treating a subject with cancer, the method comprising continuously administering to the subject an effective amount of a bis(thiohydrazide amide) compound represented by the following structural formula:

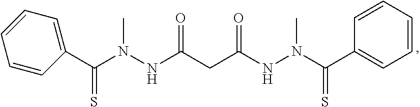 [SMILES: CN(NC(=O)CC(=O)NN(C)C(=S)c1ccccc1)C(=S)c1ccccc1]

or a pharmaceutically acceptable salt thereof, or a deprotonated form thereof complexed to a transition metal cation.

In some embodiments, the cancer is selected from the group consisting of:
i) human sarcoma or carcinoma, selected from the group consisting of fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, anal carcinoma, esophageal cancer, gastric cancer, hepatocellular cancer, bladder cancer, endometrial cancer, pancreatic cancer, breast cancer, ovarian cancer, prostate cancer, stomach cancer, atrial myxomas, squamous cell carcinoma, basal cell carcinoma, adenocarcinoma, sweat gland carcinoma, sebaceous gland carcinoma, thyroid and parathyroid neoplasms, papillary carcinoma, papillary adenocarcinomas, cystadenocarcinoma, medullary carcinoma, bronchogenic carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, pituitary neoplasms, astrocytoma, medulloblastoma, craniopharyngioma, ependymoma, pinealoma, hemangioblastoma, acoustic neuroma, schwannomas, oligodendroglioma, meningioma, spinal cord tumors, melanoma, neuroblastoma, pheochromocytoma, Types 1-3 endocrine neoplasia, retinoblastoma; and
ii) leukemia, selected from the group consisting of acute lymphocytic leukemia, acute myelocytic leukemia; chronic leukemia, polycythemia vera, multiple myeloma, Waldenstrobm's macroglobulinemia, heavy chain disease, T-cell leukemias, B cell leukemia; mixed cell leukemias, myeloid leukemias, neutrophilic leukemia, eosinophilic leukemia, monocytic leukemia, myelomonocytic leukemia, Naegeli-type myeloid leukemia, and nonlymphocytic leukemia.

In some embodiments, the cancer is selected from the group consisting of B-cell lymphoma, non-small cell lung cancer, renal cancer or colorectal cancer.

In some embodiments, the bis(thiohydrazide amide) compound is administered such that a constant concentration of the bis(thiohydrazide amide) is maintained in the subject. In one embodiment, the subject is a human.

In some embodiments, the bis(thiohydrazide amide) compound is a disalt comprising two monovalent cations M⁺ or one divalent cation M²⁺. In one embodiment, the bis(thiohydrazide amide) compound is a disalt comprising two monovalent cations M⁺. In a further embodiment, M⁺ is K⁺ or Na⁺.

In some embodiments, the bis(thiohydrazide amide) compound is a deprotonated form of the bis(thiohydrazide amide) compound complexed to a transition metal cation. In one embodiment, the transition metal cation is Ni²⁺, Cu²⁺, Co²⁺, Fe²⁺, Zn²⁺, Pt²⁺ or Pd²⁺. In a further embodiment, the transition metal cation is Cu²⁺.

In one embodiment, the bis(thiohydrazide amide) compound is administered systemically. In another embodiment, the bis(thiohydrazide amide) compound is administered subcutaneously. In another embodiment, the bis(thiohydrazide amide) compound is administered intravenously.

In one embodiment, the disclosed method of treating cancer achieves a constant concentration of the bis(thiohydrazide amide compound) in the subject being treated. In a further embodiment, the constant concentration of the bis(thiohydrazide amide) compound is plasma concentration, serum concentration, concentration at site of tumor, concentration in cells within the tumor, or concentration in the vasculature within the tumor.

In one embodiment, the bis(thiohydrazide amide) compound is continuously administered for 7 days. In a further embodiment, the bis(thiohydrazide amide) compound is continuously administered for 7 days, and the 7-day continuous administration is further repeated at least once. In another embodiment, the bis(thiohydrazide amide) compound is administered for 7 days, such that a constant concentration of the bis(thiohydrazide amide) compound is present in the subject. In a further embodiment, the bis(thiohydrazide amide) compound is administered for 7 days, such that a constant concentration of the bis(thiohydrazide amide) compound is present in the subject, and the 7-day continuous administration is further repeated at least once.

In one embodiment, the bis(thiohydrazide amide) compound is continuously administered as a monotherapy for treating cancer. In another embodiment, the bis(thiohydrazide amide) compound is continuously administered in combination with an effective amount of taxane. In a further embodiment, the taxane is paclitaxel. In another embodiment, the bis(thiohydrazide amide) compound is continuously administered in the absence of radiation, hypothermia and/or immunotherapy.

In one embodiment, the bis(thiohydrazide amide) compound is administered as a monotherapy for treating cancer, such that a constant concentration of the bis(thiohydrazide amide) is present in the subject. In another embodiment, the bis(thiohydrazide amide) compound is administered in combination with an effective amount of taxane, such that a constant concentration of the bis(thiohydrazide amide) is present in the subject. In a further embodiment, the taxane is paclitaxel. In another embodiment, the bis(thiohydrazide amide) compound is administered in the absence of radiation, hypothermia and/or immunotherapy, such that a constant concentration of the bis(thiohydrazide amide) is present in the subject.

### BRIEF DESCRIPTION OF THE DRAWINGS

FIG. 1 is a graph showing relative copper levels in the nuclear, cytosolic and mitochondrial fractions from PBMCs and HL-60 cells.

FIG. 2 is a graph showing the levels of ROS measured by the MitoSox Red assay in PBMCs and HL-60 cells treated with different concentrations of CuCl₂, DSF-Cu and elesclomol-Cu.

FIG. 3 is a graph showing cellular copper levels and elesclomol levels in PBMCs and HL-60 cells treated with a vehicle or 100 nM elesclomol.

FIG. 4 is, in part, a graph showing ⁶⁵Cu and ⁶³Cu levels measured over time in HL-60 cells treated with elesclomol-⁶⁵Cu and free ⁶⁵Cu. FIG. 4, in part, is a schematic showing the proposed mechanism of Cu shuttling by elesclomol.

FIG. 5 is a graph showing plasma elesclomol-Cu plasma concentration at various timepoints after I.V. bolus administration of elesclomol.

FIG. 6 is a graph showing % change in the body weight measured daily in the mouse toxicity study of continuously administered elesclomol.

FIG. 7 is a graph showing % organ weight/body weight for the brain, heart, kidneys, spleen, thymus, testes in the mouse toxicity study of continuously administered elesclomol.

FIG. 8 is a graph showing average tumor volume measured 26-41 days after tumor implantation of the Daudi B-Cell Lymphoma tumor in mice administered DRD or elesclomol salt by continuous infusion or administered elesclomol salt by I.V. bolus doses.

FIG. 9 is a graph showing average tumor volume measured 22-43 days after tumor implantation of the H1703 Human NSCLC tumor in mice administered DRD or elesclomol salt by continuous infusion.

FIG. 10 is a graph showing average tumor volume measured 16-37 days after tumor implantation of the 786-O RCC tumor in mice administered DRD or elesclomol salt by continuous infusion or administered elesclomol salt by I.V. bolus doses.

FIG. 11 is a graph showing average tumor volume measured 16-35 days after tumor implantation of the HCT116 human colon tumor in mice administered DRD or elesclomol salt by continuous infusion.

FIG. 12 is a graph showing average tumor volume measured 14-32 days after tumor implantation of the SW480 human colon tumor in mice administered DRD or elesclomol salt by continuous infusion.

### DETAILED DESCRIPTION OF THE INVENTION

### The Compound and Formulations Thereof

The current invention is directed to methods of treating a subject with cancer comprising continuously administering to the subject an effective amount of a bis(thiohydrazide amide) compound, or administering to the subject an amount of a bis(thiohydrazide amide) compound at intervals sufficient to maintain a constant concentration of the bis(thiohydrazide amide) compound in the subject's body, or a pharmaceutically acceptable salt thereof, or a deprotonated form thereof complexed to a transition metal ion. The bis(thiohydrazide amide) compound to be administered to the subject being treated for cancer is elesclomol represented by the following structural formula:

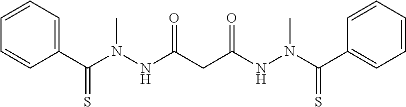 [SMILES: CN(NC(=O)CC(=O)NN(C)C(=S)c1ccccc1)C(=S)c1ccccc1]

The bis(thiohydrazide amide) compound described herein may be present in the form of a pharmaceutically acceptable salt. Pharmaceutically acceptable salt forms include pharmaceutically acceptable basic/cationic salts. Basic addition salts include those derived from inorganic bases, such as ammonium or alkali or alkaline earth metal hydroxides, carbonates, bicarbonates, and the like, and organic bases such as alkoxides, alkyl amides, alkyl and aryl amines, and the like. Such bases useful in preparing the salts of this invention thus include sodium hydroxide, potassium hydroxide, ammonium hydroxide, potassium carbonate, and the like.

In one embodiment, the bis(thiohydrazide amide) compound is in a form of a disalt represented by the following structural formula:

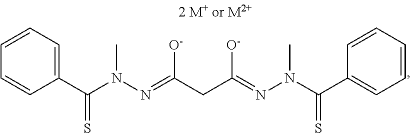

wherein M⁺ is a pharmaceutically acceptable monovalent cation and M²⁺ is a pharmaceutically acceptable divalent cation. “Pharmaceutically acceptable” means that the cation is suitable for administration to a subject. Examples of M⁺ or M²⁺ include Li⁺, Na⁺, K⁺, Mg²⁺, Ca²⁺, Zn²⁺, and NR₄⁺, wherein each R is independently hydrogen, a substituted or unsubstituted alkyl group (e.g., a hydroxyalkyl group, aminoalkyl group or ammoniumalkyl group) or substituted or unsubstituted aryl group (e.g., phenyl, naphthyl, and anthracyl, imidazolyl, thienyl, furanyl, pyridyl, pyrimidyl, pyranyl, pyrazolyl, pyrroyl, pyrazinyl, thiazole, oxazolyl, and tetrazole), or two R groups, taken together, form a substituted or unsubstituted non-aromatic heterocyclic ring optionally fused to an aromatic ring. Preferably, the pharmaceutically acceptable cation is Li⁺, Na⁺, K⁺, NH₃(C₂H₅OH)⁺, N(CH₃)₃(C₂H₅OH)⁺, arginine or lysine. More preferably, the pharmaceutically acceptable cation is Na⁺ or K⁺. Na⁺ is even more preferred.

In another embodiment, the bis(thiohydrazide amide) compound is in a deprotonated form complexed to a transition metal ion. The term “complexed” means that elesclomol attaches to the transition metal ion through one or more coordinate covalent bonds or coordination bonds. The term “chelated” means that elesclomol binds to the transition metal ion at two or more attachment points through coordinate covalent bonds or coordination bonds. The terms “coordinate”, “coordinated”, “coordinate covalent bond” and “coordination bond” have the meanings that are commonly known to one of ordinary skill in the art. A “deprotonated form” of a bis(thiohydrazide amide) compound refers to a molecule wherein one or more protons from the bis(thiohydrazide amide) compound or a salt, hydrate, solvate or polymorph thereof have been removed. A deprotonated form of the bis(thiohydrazide amide) compound is represented by the following structural formula:

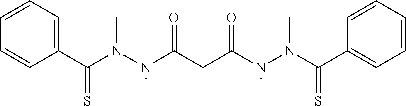 [SMILES: CN(NC(=O)CC(=O)NN(C)C(=S)c1ccccc1)C(=S)c1ccccc1]

A “transition metal cation” refers to a positively charged ion of a metal in Groups 3-12 of the Periodic Table. Examples include Ni²⁺, Cu⁺, Cu²⁺, Co²⁺, Co³⁺, Fe²⁺, Fe³⁺, Zn²⁺, Pt²⁺, Pd²⁺, V⁴⁺, V⁵⁺, Cr²⁺, Cr³⁺, Cr⁴⁺, Mn²⁺, Mn³⁺, Mn⁴⁺ and Mn⁵⁺. In a specific embodiment, the transition metal cations have a +2 charge. Examples include Ni²⁺, Cu²⁺, Co²⁺, Fe²⁺, Zn²⁺, Pt²⁺ and Pd²⁺. In a specific embodiment, the transition metal cation is Cu⁺, Cu²⁺ or Ni²⁺. In a more specific embodiment, the transition metal cation is Cu²⁺. The molar ratio of the bis(thiohydrazide amide) compound or a salt, hydrate, solvate, polymorph or a deprotonated form thereof to transition metal cation recited in this paragraph is, for example, equal to or greater than 0.5 and equal to or less than 2.0 (i.e. 0.5≦ratio≦2.0) or 1:1.

A structure of the bis(thiohydrazide amide) compound of the present invention complexed to a transition metal ion is shown below:

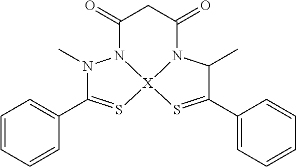

or a prodrug, isomer, ester, salt, hydrate, solvate, or polymorph thereof, wherein X is a transition metal cation having a +2 charge. In a preferred embodiment, X is Cu²⁺.

Elesclomol is preferably in a substantially pure form, e.g., greater than 50%, 60%, 70%, 80%, 90%, 95%, 97%, 99%, 99.5% or 99.9% pure by weight. “Percent purity by weight” means the weight of elesclomol divided by the weight of elesclomol plus impurities times 100%.

The bis(thiohydrazide amide) compound of the present invention can be prepared according to methods described in U.S. Pat. Nos. 6,800,660, 6,762,204, and 6,825,235, and U.S. Publication No. 2008/0146842.

The transition metal complex of the bis(thiohydrazide amide) compound of the present invention can be prepared according to methods described in WO 2010/048284 and WO 2010/048293, the entire contents of which are incorporated herein by reference.

Elesclomol may be formulated as a pharmaceutical composition also comprising a pharmaceutically acceptable carrier or diluent. As used herein, a “pharmaceutical composition” can be a formulation containing the disclosed compounds, in a form suitable for administration to a subject. Suitable pharmaceutically acceptable carriers may contain inert ingredients which do not inhibit the biological activity of elesclomol. The pharmaceutically acceptable carriers should be biocompatible, i.e., non-toxic, non-inflammatory, non-immunogenic and devoid of other undesired reactions upon the administration to a subject. Standard pharmaceutical formulation techniques can be employed, such as those described in Remington: the Science and Practice of Pharmacy, 19^{th} edition, Mack Publishing Co., Easton, Pa. (1995).

The pharmaceutical composition can be in bulk or in unit dosage form. The unit dosage form can be in any of a variety of forms, including, for example, a capsule, an IV bag, a tablet, a single pump on an aerosol inhaler, or a vial. The quantity of a bis(thiohydrazide amide) compound, e.g., elesclomol, in a unit dose is the effective amount of elesclomol, as is discussed below. The quantity of a bis(thiohydrazide amide) compound, e.g., elesclomol, in a unit dose will vary according to the chosen route of administration. A variety of routes are contemplated, including topical, oral, transmucosal or parenteral, including transdermal, subcutaneous, intravenous, intramuscular, intraperitoneal and intranasal. In one embodiment, elesclomol is administered systemically. The term “systemically administered” or “systemic administration” refers to a route of administration, wherein an agent being administered, e.g., elesclomol, distributes throughout the body before reaching the target site. Systemic administration comprises both oral and parenteral administration. This term is also meant to exclude delivery of elesclomol directly to the tumor site or to the vicinity of the tumor site.

For oral administration, elesclomol or salts thereof can be combined with a suitable solid or liquid carrier or diluent to form capsules, tablets, pills, powders, syrups, solutions, suspensions, or the like.

The tablets, pills, capsules, and the like can contain from about 1 to about 99 weight percent of the active ingredient and a binder such as gum tragacanth, acacias, corn starch or gelatin; excipients such as dicalcium phosphate; a disintegrating agent such as corn starch, potato starch or alginic acid; a lubricant such as magnesium stearate; and/or a sweetening agent such as sucrose, lactose or saccharin. When a dosage unit form is a capsule, it may contain, in addition to materials of the above type, a liquid carrier such as a fatty oil.

Various other materials can be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor, and the like.

For parental administration, elesclomol can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable salts of the compounds. Dispersions can also be prepared in glycerol, liquid polyethylene glycols and mixtures thereof in oils. Under ordinary conditions of storage and use, these preparations contain a preservative to prevent the growth of microorganisms.

In addition to the formulations previously described, elesclomol may also be formulated as a depot preparation. Suitable formulations of this type include biocompatible and biodegradable polymeric hydrogel formulations using crosslinked or water insoluble polysaccharide formulations, polymerizable polyethylene oxide formulations, impregnated membranes, and the like. Such long acting formulations may be administered by implantation or transcutaneous delivery (for example subcutaneously or intramuscularly), intramuscular injection or a transdermal patch. Thus, for example, the compounds may be formulated with suitable polymeric or hydrophobic materials, for example, as an emulsion in an acceptable oil, or ion exchange resins, or as sparingly soluble derivatives, for example, as a sparingly soluble salt.

In some embodiments of the invention, the mode of administration is by a medical device that releases the bis(thio-hydrazide amide) in vivo, e.g., the device includes a reservoir, a coating composition, a controlled release polymer matrix, or the like which comprises the bis(thio-hydrazide amide) and can release the bis(thio-hydrazide amide) in vivo. Details of releasing compounds in vivo are known in the art; see, for example, Baker, et al., “Controlled Release of Biological Active Agents”, John Wiley and Sons, 1986, the entire teachings of which are incorporated herein by reference.

Patches that are suitable for use in this invention include, for example, a matrix type patch; a reservoir type patch; a monolithic drug-in-adhesive type patch; a multi-laminate drug-in-adhesive type patch; and the like. These patches are well known in the art; see, for example, Ghosh, T. K.; Pfister, W. R.; Yum, S. I. Transdermal and Topical Drug Delivery Systems, Interpharm Press, Inc. p. 249-297, the entire teachings of which are incorporated herein by reference. One of ordinary skill in the art can determine other patches which can be employed in the present invention.

A patch can be designed to adhere to a mucous membrane surface of the subject, e.g., sublingual or buccal membrane of the oral cavity, and the like. Typically, such a patch will include a mucoadhesive that has been loaded with elesclomol. Examples of typical mucosal adhesives are described in Nagai, J. Control. Rel. (1985), 2:121-134 and in Nagai, et al., Pharm. Int. (1985), 196-200; the entire teachings of these documents is incorporated herein by reference.

The medical device can also be an implantable osmotic pump which can be used as a means for continuous infusion of elesclomol. A liquid formulation comprising elesclomol suitable for parenteral administration can be used in such device.

In addition to the formulations described above, a formulation can optionally include, or be co-administered with one or more additional drugs, e.g., other antifungals, anti-inflammatories, antibiotics, antivirals, immunomodulators, antiprotozoals, steroids, decongestants, bronchodilators, antihistamines, anticancer agents, and the like. For example, the disclosed compound can be co-administered with drugs such as such as ibuprofen, prednisone (corticosteroid) pentoxifylline, Amphotericin B, Fluconazole, Ketoconazol, Itraconazole, penicillin, ampicillin, amoxicillin, and the like. The formulation may also contain preserving agents, solubilizing agents, chemical buffers, surfactants, emulsifiers, colorants, odorants and sweeteners.

### Administration of Elesclomol for Treating Cancer

It was surprisingly discovered that elesclomol was remarkably effective at treating cancer when continuously administered as a single agent, or when administered as a single agent in such as way as to maintain a constant level of the elesclomol in a subject's body. Accordingly, the present invention provides methods for treating cancer, comprising continuously administering elesclomol to the subject in need thereof, or comprising administering elesclomol to the subject, such that the level of elesclomol in the subject's body remains constant.

The terms “continuously administered”, “administered continuously” or “continuous administration” refer to a mode of administration, wherein constant drug concentration is achieved and/or maintained in a subject. In one embodiment, the drug is elesclomol.

The term “constant drug concentration” means that a given measurement of the drug concentration in a subject is within 5%, 10%, 15% or 20% of the desired therapeutic concentration of the drug. In one embodiment, the constant drug concentration is a constant elesclomol concentration that is achieved in a subject by administration of elesclomol. In another embodiment, the constant elesclomol concentration is the concentration sufficient to achieve desired treatment objectives, e.g., achieving, partially or substantially, one or more of the following: arresting the growth or spread of a cancer, reducing the extent of a cancer (e.g., reducing size of a tumor or reducing the number of affected sites), inhibiting the growth rate of a cancer, ameliorating or improving a clinical symptom or indicator associated with a cancer (such as tissue or serum components) and/or reducing the likelihood of the cancer recurring once it has been removed or gone into remission.

In one embodiment, the constant elesclomol concentration is a constant elesclomol plasma or serum concentration. In a further embodiment, a constant plasma or serum concentration of elesclomol falls within the range of 10 ng/mL to 100 ng/mL. For example, the plasma concentration of elesclomol achieved by administering elesclomol to a subject is 10, 15, 20, 25, 30, 35, 40, 45, 50, 55, 60, 65, 70, 75, 80, 85, 90, 95 or 100 ng/mL. In other embodiments, the constant elesclomol concentration is a constant elesclomol concentration at the site of tumor, in cells within tumor, or in the vasculature within the tumor.

Maintenance of constant elesclomol concentration in a subject as discussed above can be accomplished by various treatment regiments that will depend on the chosen administration route. For example, administration of elesclomol by oral or parenteral routes can involve repeated dosings of elesclomol at predetermined time points. Alternatively, a sustained release formulation, may be administered orally, subcutaneously or transmucosally to a subject, and this administration may be repeated as often as necessary, as determined by one of skill in the art, such that the needed elesclomol concentration is achieved and maintained. Elesclomol may also be administered by infusion of a liquid formulation comprising elesclomol via a pump. The pump may be an implantable pump that delivers a solution comprising elesclomol at a predetermined rate. The concentration of elesclomol in the solution and the rate of infusion may be adjusted depending on the elesclomol levels to be achieved or on the total daily dose to be delivered to a subject.

The precise amount of elesclomol administered to a subject in a unit dose or over a given period of time will depend on the selected route of administration, the frequency of dosings, and the desired constant elesclomol levels. Effective unit doses may be extrapolated from dose-response curves derived from in vitro or animal model test systems, and by achieving certain blood levels of the drug, or by monitoring change in appropriate biomarkers that serve as a surrogate to efficacy. When extrapolating an effective unit dose from an animal model test, one of skill in the art would be familiar with the factors that can be used for converting doses expressed in terms of mg/kg from one species to an equivalent surface area dose expressed as mg/kg in another species. Such dose conversions may be carried out, e.g., by using NIH guidelines and the assumptions and constants described in Freireich E. J. et al., Quantitative comparison of toxicity of anticancer agents in mouse, rat, dog, monkey and man, Cancer Chemother. Rep. 1966, 50(4), 219-244, the entire contents of which are incorporated herein by reference. Specifically, a mouse dose of 1 mg/kg corresponds to a human equivalent dose of 12 mg/kg.

In general, the recommended daily dose range of elesclomol for the conditions described herein lie within the range of from about 0.01 mg to about 3000 mg per day. Specifically, a daily dose range should be from about 5 mg to about 500 mg per day, more specifically, between about 10 mg and about 200 mg per day. In managing the patient, the therapy should be initiated at a lower dose, perhaps about 1 mg to about 25 mg, and increased if necessary up to about 200 mg to about 1000 mg per day as either a single dose or divided doses. It may be necessary to use dosages of elesclomol outside the ranges disclosed herein in some cases, as will be apparent to those of ordinary skill in the art. Furthermore, it is noted that the clinician or treating physician will know how and when to interrupt, adjust, or terminate therapy in conjunction with individual patient response.

In some embodiments, the total daily dose of elesclomol administered to a human subject is in the range of 200-300 mg/kg, e.g., 200, 205, 210, 215, 220, 225, 230, 235, 240, 245, 250, 255, 260, 265, 270, 275, 280, 285, 290, 295 or 300 mg/kg of elesclomol. In a preferred embodiment, the total daily dose of elesclomol is 285 mg/kg. Alternatively, the total daily dose of elesclomol administered to a human subject is in the range of 300-400 mg/kg, e.g., 300, 305, 310, 315, 320, 325, 330, 335, 340, 345, 350, 355, 360, 365, 370, 375, 380, 385, 390, 395 or 400 mg/kg. Alternatively, the total daily dose of elesclomol administered to a human subject is in the range of 100-200 mg/kg, e.g., 100, 105, 110, 115, 120, 125, 130, 135, 140, 145, 150, 155, 160, 165, 170, 175, 180, 185, 190, 195 or 200 mg/kg. In various embodiments, the total daily dose of elesclomol delivered via a selected administration route is sufficient to achieve and/or maintain the desired constant elesclomol concentration in a subject.

Elesclomol is to be administered over a period of time. The period of time appropriate for continuous administration is the period of time during which achievement and/or maintenance of a constant elesclomol concentration in a subject is desired. The period of time for administration can last from 0 to 60 minutes, e.g., 5, 10, 15, 20, 25, 30, 35, 40, 45, 50, 55 or 60 minutes. Alternatively the period of time can also last from 0 to 24 hours, e.g., 1, 2, 3, 4, 5, 6, 7, 8, 9, 10, 11, 12, 13, 14, 15, 16, 17, 18, 19, 20, 21, 22, 2, or 24 hours. Alternatively, the period of time can last from 1 to 21 days, e.g., 1, 2, 3, 4, 5, 6, 7, 8, 9, 10, 11, 12, 13, 14, 15, 16, 17, 18, 19, 20, or 21 days. In one embodiment, the bis(thiohydrazide amide) compound is administered for 7 days. In another embodiment, the bis(thiohydrazide amide) compound is systemically administered for a period from 1-21 days, e.g., 1, 2, 3, 4, 5, 6, 7, 8, 9, 10, 11, 12, 13, 14, 15, 16, 17, 18, 19, 20, or 21 days.

The treatment comprising administration of a elesclomol over a certain time period can be repeated. In one embodiment, the second treatment comprising administration of elesclomol may be started immediately after the first treatment is finished. In another embodiment, the treatment may be repeated after a period of time wherein no elesclomol is administered. The appropriate interval between the two treatments will depend on the chosen route of administration and constant elesclomol concentration to be achieved by elesclomol administration, and can be determined by one of skill in the art. Ideally, the treatment is to be repeated as many times as is deemed necessary by a person prescribing or administering the treatment or until the cancer is in remission, or until the subject with cancer is treated in accordance with the description below. In one embodiment, the treatment is repeated at least once. In a further embodiment, the treatment comprises a 7-day period of elesclomol administration that is further repeated at least once.

### Methods of Treating Cancer

It was surprisingly discovered that a bis(thiohydrazide amide) compound, e.g., elesclomol displayed a remarkable single-agent activity against cancer when administered continuously, or administered such that a constant elesclomol concentration is maintained in a subject. Accordingly, the present invention provides methods of treating cancer, comprising continuously administering elesclomol to the subject in need thereof, or administering elesclomol in such a way as to maintain constant elesclomol concentration in a subject. The cancers that can be treated by the methods of the present invention are selected from the group consisting of:
i) human sarcoma or carcinoma, selected from the group consisting of fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, anal carcinoma, esophageal cancer, gastric cancer, hepatocellular cancer, bladder cancer, endometrial cancer, pancreatic cancer, breast cancer, ovarian cancer, prostate cancer, stomach cancer, atrial myxomas, squamous cell carcinoma, basal cell carcinoma, adenocarcinoma, sweat gland carcinoma, sebaceous gland carcinoma, thyroid and parathyroid neoplasms, papillary carcinoma, papillary adenocarcinomas, cystadenocarcinoma, medullary carcinoma, bronchogenic carcinoma, hepatoma, bile duct carcinoma, choriocarcinoma, seminoma, embryonal carcinoma, Wilms' tumor, cervical cancer, testicular tumor, lung carcinoma, mall cell lung carcinoma, bladder carcinoma, epithelial carcinoma, glioma, pituitary neoplasms, astrocytoma, medulloblastoma, craniopharyngioma, ependymoma, pinealoma, hemangioblastoma, acoustic neuroma, schwannomas, oligodendroglioma, meningioma, spinal cord tumors, melanoma, neuroblastoma, pheochromocytoma, Types 1-3 endocrine neoplasia, retinoblastoma; and
ii) leukemia, selected from the group consisting of acute lymphocytic leukemia, acute myelocytic leukemia; chronic leukemia, polycythemia vera, multiple myeloma, Waldenstrobm's macroglobulinemia, heavy chain disease, T-cell leukemias, B cell leukemia; mixed cell leukemias, myeloid leukemias, neutrophilic leukemia, eosinophilic leukemia, monocytic leukemia, myelomonocytic leukemia, Naegeli-type myeloid leukemia, and nonlymphocytic leukemia.

In some embodiments, the cancers that can be treated by the methods of the present invention are selected from the group consisting of B-cell lymphoma, non-small cell lung cancer, renal cancer or colorectal cancer.

As used herein, the term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human and may be referred to as a patient.

“Treating a subject with a cancer” includes achieving, partially or substantially, one or more of the following: arresting the growth or spread of a cancer, reducing the extent of a cancer (e.g., reducing size of a tumor or reducing the number of affected sites), inhibiting the growth rate of a cancer, ameliorating or improving a clinical symptom or indicator associated with a cancer (such as tissue or serum components) and/or reducing the likelihood of the cancer recurring once it has been removed or gone into remission.

The term “effective amount” is the quantity of elesclomol required to maintain constant elesclomol concentration in a subject while being effective for treating cancer. In some embodiments, the effective amount of elesclomol is sufficient to maintain the desired plasma or serum concentration of elesclomol. In other embodiments, the effective amount of elesclomol is sufficient to maintain the desired concentration of elesclomol at site or tumor, in cell within the tumor, or in the vasculature within the tumor. The precise amount of elesclomol to be administered to a subject will depend on the elesclomol levels that are to be achieved and/or maintained in a subject, as well as on the exact mode of administration, as discussed above. When elesclomol is co-administered with another anti-cancer agent, e.g., a taxane, for the treatment of cancer, an “effective amount” of the second anti-cancer agent will depend on the type of drug used. Suitable dosages are known for approved anti-cancer agents and can be adjusted by the skilled artisan according to the condition of the subject, the type of cancer being treated and the compound of the invention being used.

The terms “administer”, “administering” or “administration” include any method of delivery of a pharmaceutical composition or agent into a subject's system or to a particular region in or on a subject. In certain embodiments of the invention, an agent is administered intravenously, intramuscularly, subcutaneously, intradermally, intranasally, orally, transcutaneously, or mucosally. In a preferred embodiment, an agent is administered systemically. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, etc.

In one embodiment, elesclomol is administered as a monotherapy, e.g., as the only anticancer drug administered to a subject to treat cancer. The monotherapy with elesclomol may also comprise drugs that can be administered for indications other than cancer, e.g., antifungals, anti-inflammatories, antibiotics, antivirals, immunomodulators, antiprotozoals, steroids, decongestants, bronchodilators, antihistamines and the like. The present invention is also meant to exclude administration of elesclomol in combination with radiation, hypothermia and/or immunotherapy.

In still another embodiment, the bis-thiohydrazide amide is administered in combination with an effective amount of another anti-cancer drug, e.g., taxane. Taxanes comprise a class of anti-cancer drugs that can act by enhancing and stabilizing microtubule formation. The term “taxane” is meant to include paclitaxel (or “Taxol™”) and paclitaxel analogs. “Paclitaxel analog” is defined herein to mean a compound which has the basic paclitaxel skeleton and which stabilizes microtubule formation. Many paclitaxel analogs are known, including docetaxel (Taxotere™). Paclitaxel and docetaxel have the respective structural formulas:

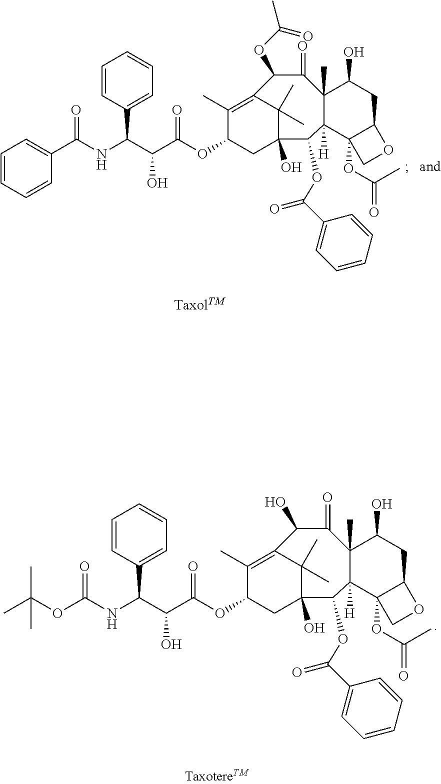

Various structural features of Taxol™ analogs have been described in previous publications, e.g., US 2009/0137682, the entire contents of which are incorporated herein by reference.

The dosages of taxanes to be administered with the bis(thiohydrazide amide) compound in accordance with the methods of the present invention will depend on the subject to be treated and the severity of the disease. The recommended dosages of taxanes can obtained from any reference in the art including, but not limited to, Hardman et al., eds., 1996, Goodman & Gilman's The Pharmacological Basis Of Basis Of Therapeutics 9^{th} Ed, Mc-Graw-Hill, New York; Physician's Desk Reference (PDR) 57^{th} Ed., 2003, Medical Economics Co., Inc., Montvale, N.J.

When the bis(thiohydrazide amide) compound of the invention is administered in combination with a taxane, the therapies can be administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours apart.

The present invention is illustrated by the following examples, which are not intended to be limiting in any way.

### EXAMPLES

Elesclomol is a first-in-class investigational drug that exerts potent anticancer activity through the elevation of reactive oxygen species (ROS) levels and is currently under clinical evaluation as a novel anticancer therapeutic. Elesclomol preferentially binds extracellular copper (Cu) and selectively transports this metal ion to the mitochondria of tumor cells to promote mitochondrial ROS generation and subsequent apoptosis.

### Example 1

### Elesclomol-Cu Complex Selectively Increases Cu Levels in the Mitochondria of Cancer Cells

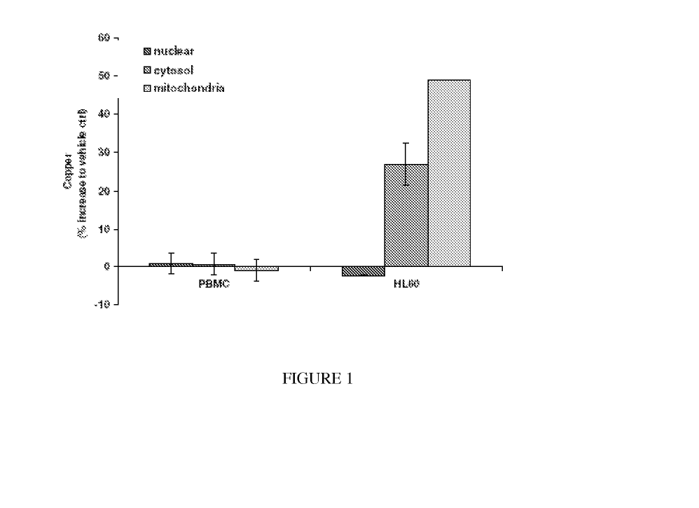

Human peripheral blood mononuclear cells (PBMCs) from 3 independent donors and human promyelotic tumor cell line HL-60 were treated with the elesclomol-Cu complex for 2 hours. Cells were subsequently fractionated into cytosolic, nuclear or mitochondrial fractions and the subcellular copper content was determined by BCA assay. FIG. 1 shows relative copper levels in the nuclear, cytosolic and mitochondrial fractions from PBMCs and HL-60 cells. Despite similar cellular uptake of elesclomol by both cell types, copper levels increased only in HL-60 cells, particularly, in the mitochondrial fraction.

### Example 2

### Elesclomol-Cu Complex Selectively Increases ROS Levels in the Mitochondria of Cancer Cells

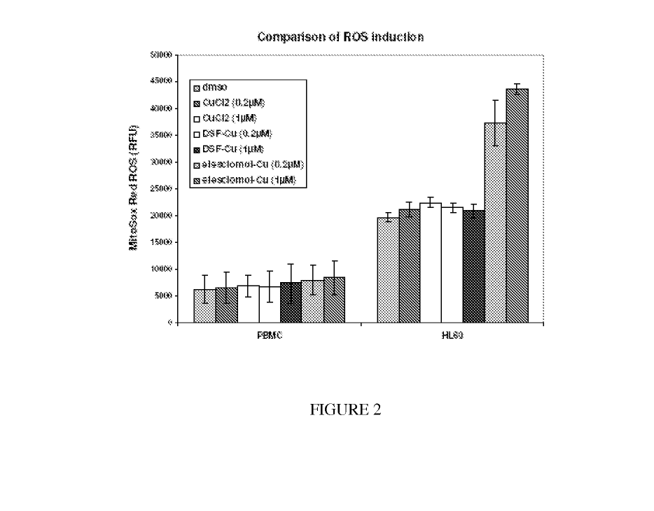

Mitochondria isolated from PBMCs or from HL-60 cells were treated for 30 minutes with either DMSO, free copper (CuCl₂) at the concentrations of 0.2 μM and 1 μM, disulfiram-Cu complex (DSF-Cu) at the concentrations of 0.2 μM and 1 μM, and elesclomol-Cu complex at the concentrations of 0.2 μM and 1 μM. Subsequent to incubation, ROS levels were measured by MitoSox Red. The results of the MitoSox Red assay are shown in FIG. 2 as mean±SD of experiments with PBMCs isolated from three independent donors or with three individual experiments with HL-60. The data demonstrate that elesclomol-Cu induces ROS in HL-60-derived mitochondria but not in those isolated from PBMCs. These results suggest that elesclomol-Cu selectively targets cancer cell mitochondria to ultimately produce critical elevations in oxidative stress.

### Example 3

### Selective Elevation of Cu Levels in Cancer Cells is not Caused by the Differential Update of Elesclomol

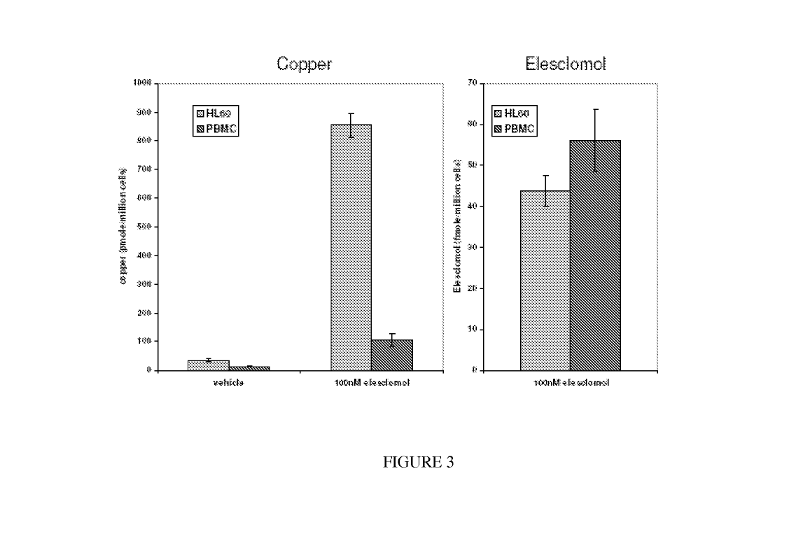

PBMCs and HL-60 cells were incubated with a vehicle (DMSO) or with 100 nM elesclomol for 3 hours. Subsequent to incubation, cellular copper levels and elesclomol levels for PBMCs and HL-60 cells were determined and are shown in FIG. 3. The results demonstrate that copper levels in HL-60 cells are remarkably and selectively elevated in response to elesclomol treatment while elesclomol levels in HL60 cells are slightly lower than that in PBMCs. The results suggest that this increase is not caused by the differential uptake of elesclomol by PBMCs and HL-60 cells.

### Example 4

### Length of Elesclomol Exposure is Proportional to the Amount of Cu Accumulated in Cancer Cells

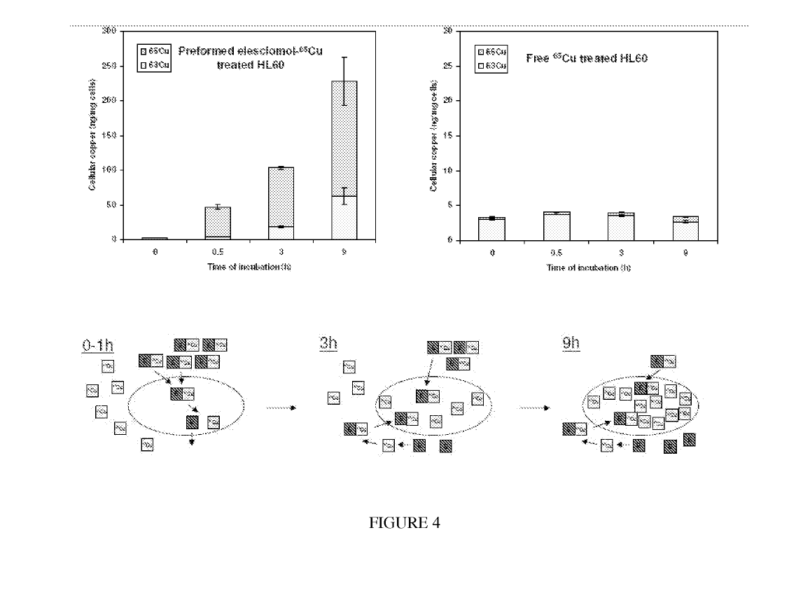

A complex of elesclomol with ⁶⁵Cu was prepared. HL-60 cells enriched with ⁶³Cu were treated with 100 nM elesclomol-⁶⁵Cu or 100 nM free ⁶⁵Cu for 0, 0.5, 3 and 9 hours in 10% FBS medium. Cellular ⁶³Cu and ⁶⁵Cu levels were measured by inductively coupled plasma mass spectroscopy (ICP-MS). The results of these measurements are shown in FIG. 4 and suggest that elesclomol-⁶⁵Cu complex, but not free ⁶⁵Cu, cause accumulation of ⁶³Cu in HL-60 cancer cells. The results also demonstrate that the amount of copper accumulated in the cells is directly proportional to the length of time that HL-60 cells are exposed to ⁶⁵Cu-elesclomol complex. These results suggest a potential benefit of continuous Cu-elesclomol exposure in vivo.

### Example 5

### Pharmacokinetic Profiles of Elesclomol and Elesclomol-Cu Complex Administered as an I.V. Bolus Dose and as Continuous Infusion

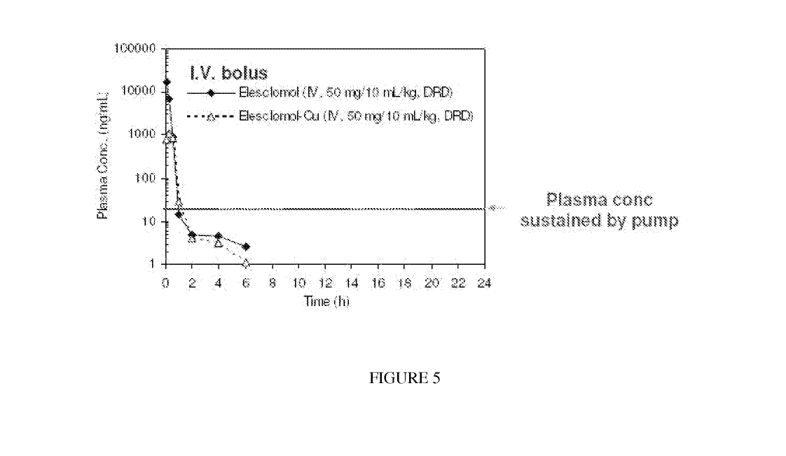

Mice received elesclomol salt administered in a single I.V. bolus dose of 50 mg/kg. Mice also received elesclomol salt administered by continuous infusion via Alzet pump. The pump was filled with a 40 mg/mL solution of elesclomol salt, and the solution was administered at 1 ml/hr, for a total daily dose of 32 mg/kg/day, based on average body weight of 30 g. The solution was delivered continuously for 7 days, and exposure was calculated from plasma levels collected at 70 hours post pump implantation. FIG. 5 shows plasma elesclomol levels (total elesclomol and elesclomol-Cu) at various time points post bolus injection as well as the elesclomol-Cu plasma concentration at 70 hours post pump implantation. The results indicate that I.V. bolus administration results in an initial high Cₘₐₓ, followed by rapid elimination. This is in stark contrast to the sustained elesclomol-Cu levels achieved by continuous infusion. Table 1 shows daily exposure to total elesclomol and elesclomol-Cu following I.V. bolus administration and continuous infusion in mice, and 1-hour infusion in humans. Similar to I.V. bolus injection in mice, 1-hour infusion in humans results in a high level of total elesclomol with an approximately 10-fold lower level of elesclomol-Cu complex. The sustained elesclomol-Cu levels achieved by continuous administration in mice demonstrate therapeutic benefit for extending the infusion duration when treating cancer patients.

TABLE 1
Total daily exposure to elesclomol-Cu following I.V. bolus
administration and continuous infusion.

| Exposure/day (h * ng/mL/d) | | Total elesclomol | Elesclomol-Cu |
|---|---|---|---|
| Mouse | Continuous infusion | 1183 | 516 |
| | (pump); 32 mg/kg/d | 5140 | 670 |
| | I.V. bolus; 50 mg/kg | | |
| Human | 1-hour infusion; 200 mg/m² | 4938 | 358 |

### Example 6

### Mouse Toxicity Study of Elesclomol Salt Continuously Administered via Alzet Pump

A total of 15 mice of strain CD1 were used for the toxicity study, of which 10 were in the elesclomol salt treatment group, and 5 were in the control group. Alzet pump was filled with a total of 200 μl of infusion solution and implanted subcutaneously within 1-2 hours. The infusion solution was continuously delivered for 7 days, and the pump was replaced with a new pump at day 8. The details of the continuous dosing are summarized in Table 2 below.

TABLE 2
Dosing of elesclomol salt for mouse toxicity study.

| | Estimated | | | |
|---|---|---|---|---|
| | dose levels | | Delivery | |
| | (assumed | Dosing | rate and | |
| Tox and | body weight | concentrations | duration of | Treatment |
| TK¹ groups | of 25-30 g) | (mg/ml) | Alzet pump | duration |
| Vehicle | 10% DMSO/ | — | 1 μl/hr, | 2 weeks |
| | 18% Cr-RH40/ | | 7 days | |
| | D5W | | | |
| Elesclomol | 32-36 mg/kg/ | 40 mg/ml | | |
| salt | d × 14 | | | |

¹toxicokinetics

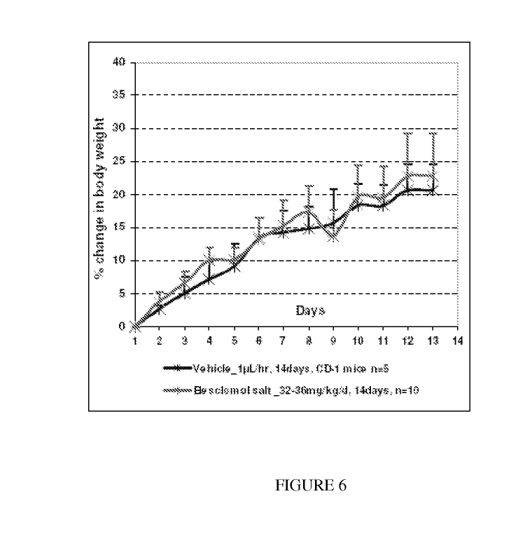

FIG. 6 shows % change in the body weight measured daily during the duration of treatment, and indicates that there are no differences between the treatment and the control groups. The results demonstrate that there are no changes in body weight caused by the continuously infused elesclomol salt.

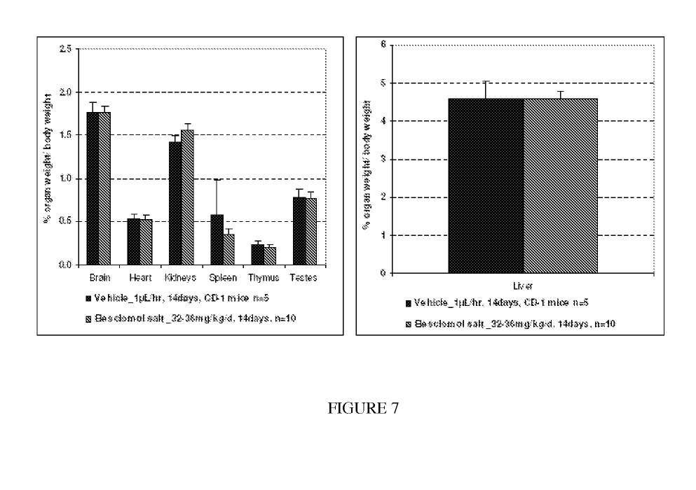

FIG. 7 shows % organ weight/body weight for the brain, heart, kidneys, spleen, thymus, testes and liver and indicates that there are no differences between the treatment and the control groups for all tested organs with the exception of spleen. Modest increases in spleen weight were associated with reactive changes in the implantation site observed in vehicle-treated animals. The results demonstrate that there are no changes in organ weight caused by the continuously infused elesclomol salt.

Table 3 below summarizes further observations of toxicity-related parameters that were assessed for the treatment and control groups. These observations indicate absence of adverse effects associated with continuous administration of elesclomol salt.

TABLE 3
Toxicity-related parameters measured for control and treatment groups.

| Treatment Groups | Vehicle | Elesclomol salt |
|---|---|---|
| Route of administration (pump | SC (Alzet pump days 1 | SC (Alzet pump days 1 and |
| implantation) | and 8) | 8) |
| Mortality (male CD1-mice) | 0/5 | 0/10 |

Comparison among groups below: G2 vs. G1

| | | |
|---|---|---|
| Max % ↓ body weight gain | — | −1.95 (day 9, 1 day after 2^{nd} |
| | | implantation) |
| Change in pupil color | — | — |
| Clinical observations | — | — |
| Necropsy: reactive change at | 1/5(+), 2/5(+++) | 1/10(++) |
| implantation site | | |
| Spleen weight: | Increase of 39% | No TA related changes |
| Clinical pathology tests | Consistent with reactive | No TA related changes |
| | change | |
| Histopathology evaluation: (brain, | Liver: consistent with | No TA related changes |
| liver and kidneys) | reactive change | |
| Plasma exposure | n.d. | Average 567 h * ng/mL/d |
| Serum ceruloplasmin (control: 1.1 ± | 1.5 ± 0.44 mg/ml | 1.3 ± 0.32 mg/ml |
| 0.3 mg/ml) | | |

The results of the toxicity study indicate that continuous administration of elesclomol is not associated with acute toxicity in mice.

### Example 7

### Single-Agent Anti-Cancer Activity of Continuously Administered Elesclomol

Anti-cancer activity of continuously administered elesclomol was evaluated using human tumor xenograft models in Nude mice. Specifically, nude mice bearing tumors derived from Daudi B-cell lymphoma, H1703 non-small cell lung cancer, 786-O renal cancer, HCT116 colorectal cancer and SW480 colorectal cancer were used for the experiments. A total of 6 mice per each tumor type were used. For continuous administration, Alzet pump was filled with 40 mg/mL solution of elesclomol salt, and the solution was delivered at 1 μl/hr for 7 days. A fresh pump was replaced every week. The daily dose of elesclomol salt administered to mice by continuous infusion was 32 mg/kg/day, based on average body weight of 30 g. The vehicle control group received Alzet pumps filled with 10% DMSO/18% Cr-RH40/D5W (DRD). This treatment was compared with the treatment comprising 5 days administration per week of elesclomol salt at 75 mg/kg, delivered by bolus I.V. injection.

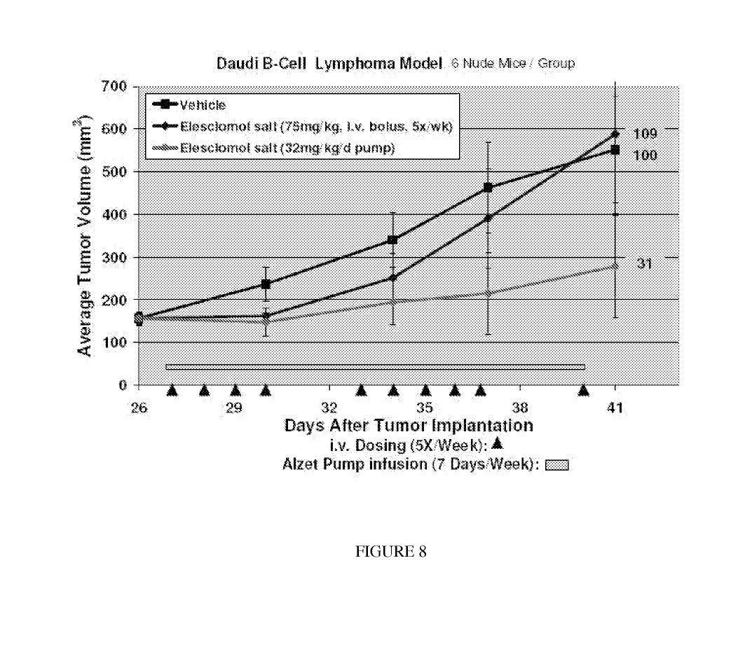

FIG. 8 shows average tumor volume measured 26-41 days after tumor implantation of the Daudi B-Cell Lymphoma tumor in mice administered DRD or elesclomol salt by continuous infusion or administered elesclomol salt by I.V. bolus doses.

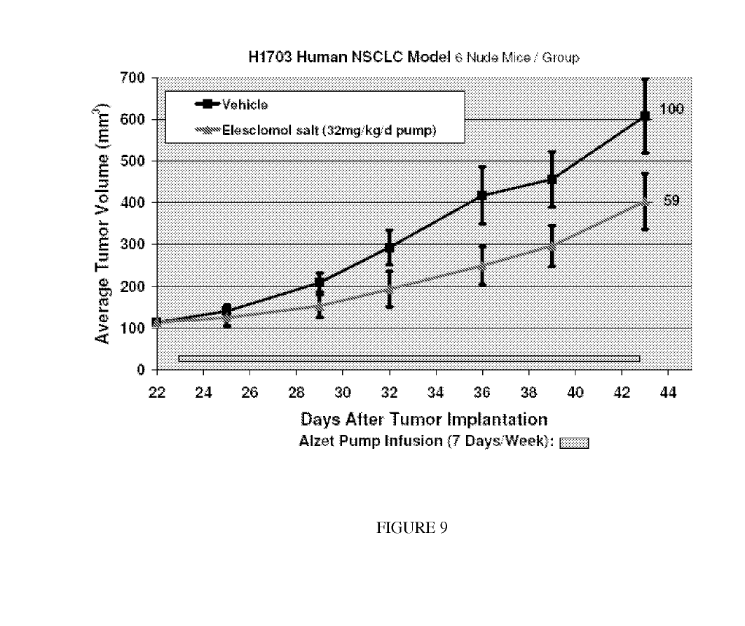

FIG. 9 shows average tumor volume measured 22-43 days after tumor implantation of the H1703 Human NSCLC tumor in mice administered DRD or elesclomol salt by continuous infusion.

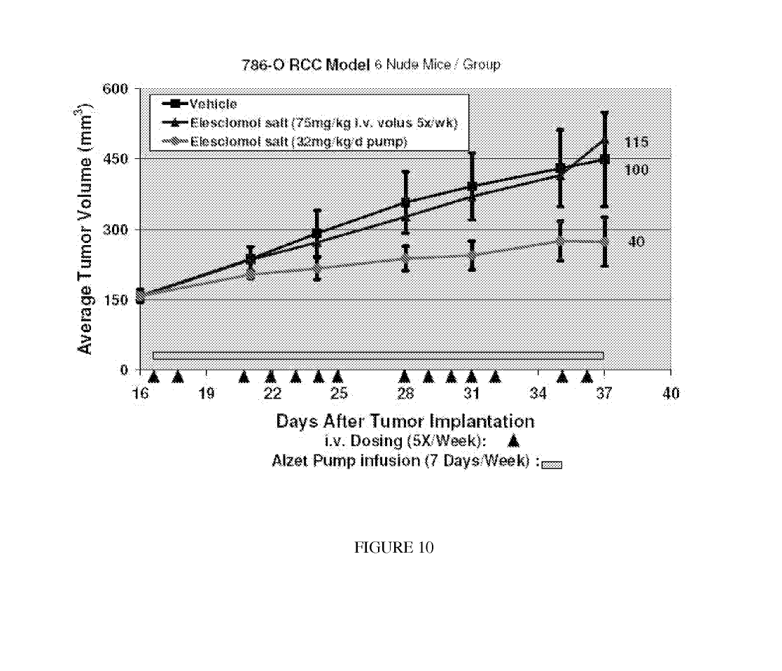

FIG. 10 shows average tumor volume measured 16-37 days after tumor implantation of the 786-O RCC tumor in mice administered DRD or elesclomol salt by continuous infusion or administered elesclomol salt by I.V. bolus doses.

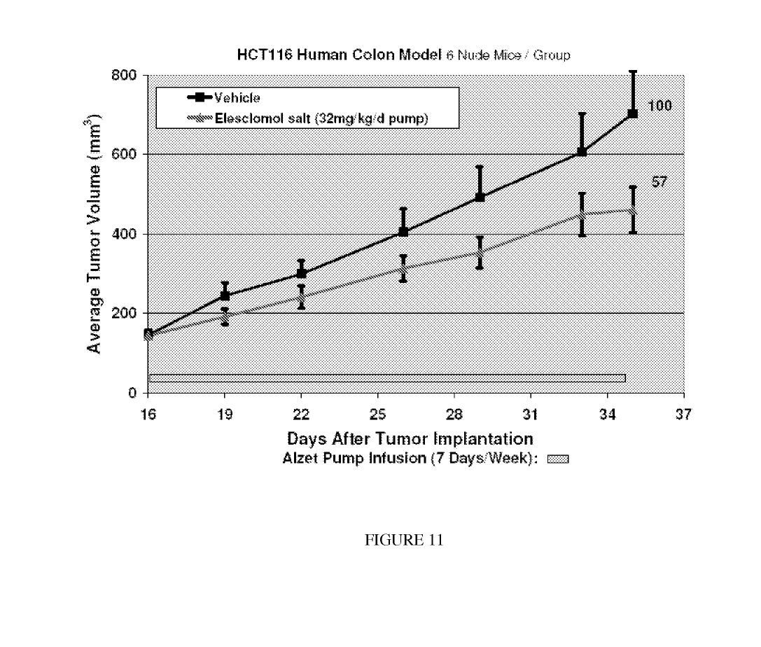

FIG. 11 shows average tumor volume measured 16-35 days after tumor implantation of the HCT116 human colon tumor in mice administered DRD or elesclomol salt by continuous infusion.

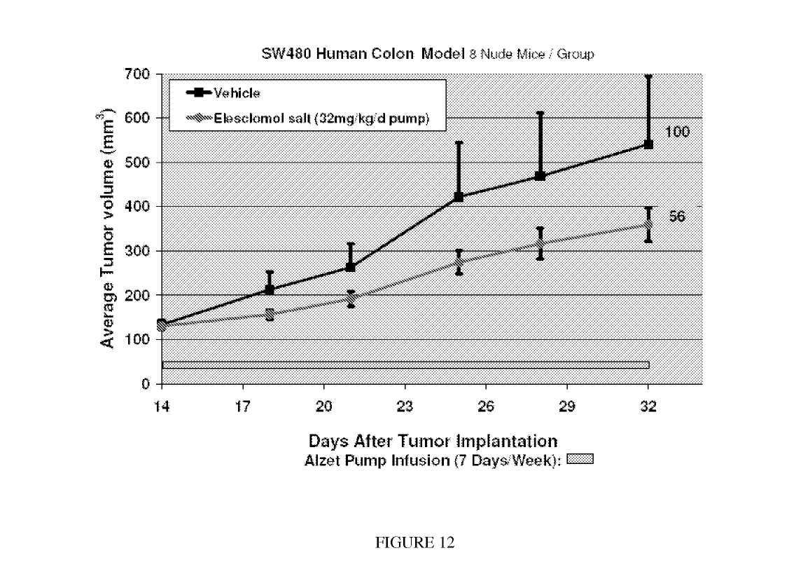

FIG. 12 shows average tumor volume measured 14-32 days after tumor implantation of the SW480 human colon tumor in mice administered DRD or elesclomol salt by continuous infusion.

Table 4 below summarizes the results shown in FIGS. 8-12 expressed as % control. The data demonstrates that continuous infusion of elesclomol salt results in inhibition of tumor growth for a variety of cancer models, and that this inhibition is far more pronounced as compared to daily bolus injection of elesclomol salt.

TABLE 4
Anti-cancer single agent activity of continuously administered
elesclomol salt.

| | | % T/C | | |
|---|---|---|---|---|
| | | Elesclomol Salt | | DRD |
| | | 32 mg/kg/d | 75 mg/kg I.V. | (control) |
| Tumor | | Alzet pump | bolus 5x/week | Alzet pump |
| Daudi | B cell | 31 | 109 | 100 |
| | lymphoma | | | |
| H1703 | NSCLC | 59 | — | 100 |
| 786-O | Renal cancer | 40 | 115 | 100 |
| HCT116 | Colorectal | 57 | — | 100 |
| SW480 | cancer | 56 | — | 100 |

1. A method of treating a subject with cancer, the method comprising continuously administering to the subject an effective amount of a bis(thiohydrazide amide) compound represented by the following structural formula: 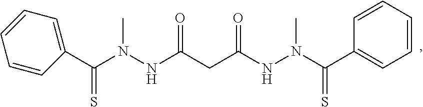 [SMILES: CN(NC(=O)CC(=O)NN(C)C(=S)c1ccccc1)C(=S)c1ccccc1]
or a pharmaceutically acceptable salt thereof, or a deprotonated form thereof complexed to a transition metal cation.

2. The method of claim 1, wherein the cancer is selected from the group consisting of:
i) human sarcoma or carcinoma, selected from the group consisting of fibrosarcoma, myxosarcoma, liposarcoma, chondrosarcoma, osteogenic sarcoma, chordoma, angiosarcoma, endotheliosarcoma, lymphangio sarcoma, lymphangioendotheliosarcoma, synovioma, mesothelioma, Ewing's tumor, leiomyosarcoma, rhabdomyosarcoma, anal carcinoma, esophageal cancer, gastric cancer, hepatocellular cancer, bladder cancer, endometrial cancer, pancreatic cancer, breast cancer, ovarian cancer, prostate cancer, stomach cancer, atrial myxomas, squamous cell carcinoma, basal cell carcinoma, adenocarcinoma, sweat gland carcinoma, sebaceous gland carcinoma, thyroid and parathyroid neoplasms, papillary carcinoma, papillary adenocarcinomas, cystadenocarcinoma, medullary carcinoma, bronchogenic carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, pituitary neoplasms, astrocytoma, medulloblastoma, craniopharyngioma, ependymoma, pinealoma, hemangioblastoma, acoustic neuroma, schwannomas, oligodendroglioma, meningioma, spinal cord tumors, melanoma, neuroblastoma, pheochromocytoma, Types 1-3 endocrine neoplasia, retinoblastoma; and
ii) leukemia, selected from the group consisting of acute lymphocytic leukemia, acute myelocytic leukemia; chronic leukemia, polycythemia vera, multiple myeloma, Waldenstrobm's macroglobulinemia, heavy chain disease, T-cell leukemias, B cell leukemia; mixed cell leukemias, myeloid leukemias, neutrophilic leukemia, eosinophilic leukemia, monocytic leukemia, myelomonocytic leukemia, Naegeli-type myeloid leukemia, and nonlymphocytic leukemia.

3. The method of claim 1, wherein the cancer is selected from the group consisting of B-cell lymphoma, non-small cell lung cancer, renal cancer or colorectal cancer.

4. The method of claim 1, wherein the bis(thiohydrazide amide) compound is a disalt comprising two monovalent cations M⁺ or one divalent cation M²⁺.

5. The method of claim 4, wherein the bis(thiohydrazide amide) compound is a disalt comprising two monovalent cations M⁺.

6. The method of claim 5, wherein M⁺ is K⁺ or Na⁺.

7. The method of claim 1, wherein the bis(thiohydrazide amide) compound is a deprotonated form of the bis(thiohydrazide amide) compound complexed to a transition metal cation.

8. The method of claim 7, wherein the transition metal cation is Ni²⁺, Cu²⁺, Co²⁺, Fe²⁺, Zn²⁺, Pt⁺ or Pd²⁺.

9. (canceled)

10. The method of claim 1, wherein the subject is a human.

11. The method of claim 1, wherein the bis(thiohydrazide amide) compound is administered systemically.

12. The method of claim 1, wherein the bis(thiohydrazide amide) compound is administered subcutaneously.

13. The method of claim 1, wherein the bis(thiohydrazide amide) compound is administered intravenously.

14. The method of claim 1, wherein the constant concentration of the bis(thiohydrazide amide compound) is achieved in the subject.

15. The method of claim 1, wherein the constant concentration of the bis(thiohydrazide amide) compound is plasma concentration, serum concentration, concentration at site of tumor, concentration in cells within the tumor, or concentration in the vasculature within the tumor.

16. The method of claim 1, wherein the bis(thiohydrazide amide) compound is continuously administered for 7 days.

17. The method of claim 1, wherein the bis(thiohydrazide amide) compound is continuously administered for 7 days, and wherein the 7-day continuous administration is further repeated at least once.

18. The method of claim 1, wherein the bis(thiohydrazide amide) compound is administered as a monotherapy for treating cancer.

19. The method of claim 1, wherein the bis(thiohydrazide amide) compound is administered in combination with an effective amount of a taxane.

20. The method of claim 19, wherein the taxane is paclitaxel.

21. The method of claim 1, wherein the bis(thiohydrazide amide) compound is administered in the absence of radiation, hypothermia and/or immunotherapy.

22-42. (canceled)
